SEQUENCE LISTING (7 sequences)

SEQ ID NO: 1 - DNA, 21 bp, Artificial Sequence
  Description of Artificial Sequence Primer
gccaaagtcttgattgattgg

SEQ ID NO: 2 - DNA, 20 bp, Artificial Sequence
  Description of Artificial Sequence Primer
ttgaagttctccagctcctg

SEQ ID NO: 3 - DNA, 20 bp, Artificial Sequence
  Description of Artificial Sequence Primer
tggatacgtttccttataag

SEQ ID NO: 4 - DNA, 19 bp, Artificial Sequence
  Description of Artificial Sequence Primer
gaaatggaggcaccccttc

SEQ ID NO: 5 - protein, 23 aa, Unknown Organism
  Description of Unknown Organism TLR-4 peptide
FKEIRHKLTLRNNFDLSLNVMKT

SEQ ID NO: 6 - DNA, 2400 bp, Homo sapiens
  CDS (1)..(2397)
atggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctga

SEQ ID NO: 7 - protein, 799 aa, Homo sapiens
MELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

(19) United States
(12) Patent Application Publication
(10) Pub. No.: US 2003/0077279 A1
(43) Pub. Date: Apr. 24, 2003

(54) Methods for treating vascular disease by inhibiting toll-like receptor-4

(75) Inventors: Moshe Arditi, Encino, CA (US); Tripathi Rajavashisth, El Camino Village, CA (US); Prediman K. Shah, Los Angeles, CA (US)

Correspondence Address: Pillsbury Winthrop LLP, Intellectual Property Group, Suite 2800, 725 South Figueroa Street, Los Angeles, CA, 90017-5406, US

(73) Assignee: CEDARS-SINAI MEDICAL CENTER

(21) Appl. No.: 10/128,166

(22) Filed: Apr. 23, 2002

Related U.S. Application Data
(60) Provisional application No. 60/341,359, filed on Dec. 17, 2001.
(60) Provisional application No. 60/335,637, filed on Oct. 24, 2001.

(51) Int. Cl. A61K 48/00; A61K 39/395; A61M 31/00

(52) U.S. Cl. 424/144.1; 514/44; 604/500

(57) ABSTRACT

Methods included herein describe the treatment of atherosclerosis and other vascular diseases such as thrombosis, restenosis after angioplasty and/or stenting, and vein-graft disease after bypass surgery, by inhibition of the expression or biologic activity of Toll-like receptor-4 (TLR-4). Also included is an intravascular device coated with a compound that inhibits TLR-4; thereby imparting an improved efficacy to the device. TLR-4 cell signal transduction is at least partially responsible for the manifestation, continuation, and/or worsening of atherosclerosis and other forms of vascular disease. The present invention provides several means with which to inhibit this signal transduction pathway.

This application claims the benefit of priority under 35 U.S.C. §119 of provisional application 60/335,637, filed Oct. 24, 2001, and provisional application 60/341,359, filed Dec. 17, 2001, the contents of which are hereby incorporated by reference.

### FIELD OF THE INVENTION

This invention relates to methods for inhibiting the biological activity of Toll-like receptor-4 (“TLR-4”), and, in particular, to methods for treating vascular disease by inhibiting the expression or signaling by TLR-4.

### BACKGROUND OF THE INVENTION

Heart disease remains the leading cause of death worldwide, accounting for nearly 30% of the annual total (i.e., approximately 15 million people). Heart and vascular disease debilitate many more individuals every year. For many, atherosclerotic disease is a life-long process; it may possess an initial stage in childhood, without clinical manifestation until middle age or later. Its development has been repeatedly linked to unhealthy lifestyles (e.g., tobacco use, unbalanced diet, and physical inactivity). Much progress has been made in the detection and treatment of various forms of heart and vascular disease, but preventative measures and assorted treatment regimens are usually incapable of halting or curing the underlying disease condition.

Experimental work over the past decade has linked inflammation of the blood vessel wall to atherogenesis, restenosis, and plaque disruption. The precise triggers for inflammation are not known, but it is believed that some triggers may include modified lipoproteins and various local or distant infections. A potential role for infection in the development of atherosclerosis has been considered; specific infectious agents, such as Chlamydia pneumoniae (“C. pneumoniae”), have been suggested as playing a role in the progression and/or destabilization of atherosclerosis.

Recent studies suggest that chlamydia lipopolysaccharide (“cLPS”) induces foam-cell formation, whereas its heat-shock protein (“cHSP-60”) induces oxidative modification of low-density lipoproteins (“LDL”). M. V. Kalayoglu and G. I. Byrne, “Chlamydia pneumoniae component that induces macrophage foam cell formation is Chlamydia lipopolysaccharide,” Infect. & Immunity 66:5067-5072 (1998); G. I. Byrne and M. V. Kalayoglu, “Chlamydia pneumoniae and atherosclerosis: Links to the disease process,” Amer. Heart Journal 138:S488-S490 (1999). cHSP-60 has been implicated in the induction of deleterious immune responses in human chlamydial infection and has been found to co-localize with infiltrating macrophages in atheroma lesions. A. G. Kol et al., “Chlamydial heat shock protein 60 localizes in human atheroma and regulates macrophage tumor necrosis factor alpha and matrix metalloproteinase expression,” Circulation 98:300 (1998). Collectively, these data support a potential role for C. pneumoniae in the development and progression of atherosclerosis and suggest that this organism may indeed play an active role in atheroma development. However, available data underscore the current lack of an understanding of the molecular mechanisms that link C. pneumoniae infections to innate immunity and trigger the signals for enhanced inflammation and atherogenesis. Absent such an understanding, it is quite difficult to develop a useful mechanism for treating vascular disease based on these data.

Although precise triggers for inflammation in atherosclerosis are not fully understood, hypercholesterolemia, modified lipoproteins, and infection with organisms such as C. pneumoniae and others have been implicated. There is evidence that C. pneumoniae infection can accelerate the progression and facilitate the induction of atherosclerosis in cholesterol-fed rabbits and genetically modified atherosclerosis prone mice. Without a clear understanding of the mechanism that controls this system, however, these data may not provide the basis for a treatment or cure for atherosclerosis. J. B. Muhlestein et al., “Infection with Chlamydia pneumoniae accelerates the development of atherosclerosis and treatment with azithromycin prevents it in a rabbit model,” Circulation 97:633-636 (1998); T. C. Moazed et al., “Murine models of Chlamydia pneumoniae infection and atherosclerosis,” J. Infect. Dis. 175:883-890 (1997); T. C. Moazed et al., “Chlamydia pneumoniae infection accelerates the progression of atherosclerosis in Apolipoprotein E-deficient mice,” J. Infect. Dis. 180:238-241 (1999); L. A. Campbell and C. C. Kuo, “Mouse models of Chlamydia pneumoniae infection and atherosclerosis,” Am. Heart J. 138:S516-S518 (1999); K. Laitinen et al., “Chlamydia pneumoniae infection induces inflammatory changes in the aortas of rabbits,” Infect & Immunity 65:4832-4835 (1997).

The concept of C. pneumoniae-induced atherogenesis is strengthened by the finding that antibiotic therapy against chlamydia prevents acceleration of atherosclerosis in the rabbit model. Ingalls et al. have suggested lipopolysaccharide (“LPS”), and Kol et al. have implicated HSP-60 as the triggers for chlamydia-induced inflammatory responses. R. R. Ingalls et al., “The inflammatory cytokine response to Chlamydia trachomatis infection is endotoxin mediated,” Infect & Immun. 63:3125-3130 (1995); A. Kol et al., “Chlamydial and human heat shock protein 60s activate human vascular endothelium, smooth muscle cells and macrophages,” J Clin Invest 103:571-577 (1999); A. Kol et al., “Heat shock protein (HSP)60 activates the innate immune response,” The J of Immunol. 164:13-17 (2000). To date, however, the precise molecular mechanisms by which infections such as C. pneumoniae contribute to the progression of atherosclerosis and the links among lipids, microbial antigens, and innate immune and inflammatory responses are not well understood.

One recent study, however, indicated that HSP-60 induces smooth muscle cell proliferation in vitro; smooth muscle cell proliferation being directly related to atherogenesis. Sasu et al., “Chlamydia pneumoniae and Chlamydial Heat Shock Protein 60 Stimulate Proliferation of Vascular Smooth Muscle Cells via Toll-Like Receptor 4 and p44/p42 Mitogen-Activated Protein Kinase Activation,” Circ. Res. 89:244-250 (2001). The study showed that smooth muscle cell proliferation was blocked or severely hampered by anti-TLR-4 antibodies. This finding suggests that HSP-60 also causes smooth muscle cell proliferation via a TLR-4 pathway.

The introduction of surgical and percutaneous arterial revascularization to treat atherosclerosis has profoundly altered the clinical management of disease, but has also brought unanticipated problems and unanswered questions. Surgical, and especially percutaneous revascularization, may elicit an exaggerated healing response, which in many respects is similar to the development of de novo atherosclerotic lesions. This “response to injury” is more proliferative in nature than de novo lesion formation, but may nevertheless lead to restenosis, or even late or abrupt vessel closure, and may ultimately result in a failed revascularization attempt. For this and additional reasons, long-term clinical studies have documented improved outcomes only in select patient subgroups; for those with stable angina pectoris, coronary intervention remains merely palliative, and does not alter the progression or outcome of the underlying causative disease process.

With balloon coronary angioplasty, restenosis rates of 30%-40% or more have been documented, and certain lesion sites and patient subgroups have been found to be particularly susceptible to restenosis. Intensive research efforts into the cause of restenosis have yielded considerable insight, but as yet no unequivocal treatment has been identified to eliminate the problem. Technical innovations in revascularization equipment and techniques have shown some success, but even this has been of limited efficacy. In particular, the development of the intracoronary stent markedly reduced the incidence of restenosis. With proper stent placement techniques, restenosis rates have been reduced to roughly 15%-30%, so intracoronary stent placement has largely supplanted balloon angioplasty alone as the interventional coronary treatment of choice. Still, given the rapid proliferation and acceptance of intracoronary stenting, even a 15%-30% restenosis rate results in a very large number of patients in whom the revascularization attempt has been unsuccessful, and for whom other treatment strategies have not been sufficiently effective. Often, the same patient may need multiple separate interventions, and ultimately these may not be successful.

Since the arterial response to injury is predominantly mitogenic and neoproliferative in nature, intracoronary irradiation (or intracoronary brachytherapy) has been developed and deployed to attempt to reduce further the number of patients who restenose following coronary intervention. Intracoronary brachytherapy has also met with limited success, however, and has brought with it two new manifestations of the disease as a side effect: geometric miss and late in-stent thrombosis. It appears likely that these two effects will significantly limit the efficacy of intracoronary brachytherapy as a definitive treatment for restenosis. Thus, a need remains for an effective way to limit or eliminate restenosis following coronary stent placement. Alternatively, if intracoronary brachytherapy is to achieve unequivocal effectiveness in eliminating restenosis following stent placement, a solution to late in-stent thrombosis and geometric miss must be found.

Conventional treatments for vascular disease have substantial drawbacks; many are only partially effective, and few provide a true cure for associated conditions. There remains a clear need in the art for a method of preventing, treating, and curing vascular disease, including atherosclerosis. There remains a further need in the art for improvements to present stent technology, whereby one can minimize the chance of restenosis.

### SUMMARY OF THE INVENTION

It is an object of the present invention to provide methods for inhibiting the biological activity of TLR-4, as, for example, by inhibiting its expression or signaling. It is a further object of the invention to provide methods of treating those diseases in which inhibiting TLR-4 would have a beneficial effect. Such diseases include, for example, vascular disease such as atherosclerosis and thrombosis, restenosis after angioplasty and/or stenting, and vein-graft disease after bypass surgery.

A first embodiment of the invention is directed to a method of inhibiting TLR-4 by administering to a mammal recombinant viral vectors (e.g., adenovirus, adeno-associated virus, retroviruses, lentiviruses, or other viral vectors) that deliver genes expressing antisense TLR-4 RNA; doing so inhibits the expression of TLR-4, thereby inhibiting its biological activity. An optimal amount of viral particles and an effective and convenient route to administer it (e.g., by administering it intravenously or intramuscularly) can readily be determined by one of ordinary skill in the art of microbiology.

A second embodiment of the present invention is directed to a method of inhibiting TLR-4 signaling by inducing in vivo production of a high affinity soluble TLR-4 protein that competes for non-bound HSP-60, LPS, and other ligands that are molecularly configured to operatively interact with a TLR-4 receptor. The TLR-4 protein most preferably lacks the TLR-4 signal transduction domain, or at least a sufficient amount of the TLR-4 signal transduction domain such that the TLR-4 protein is unable to participate in TLR-4 signal transduction. The method involves delivering viral vectors to produce an amount of soluble TLR-4 or its derivatives that is sufficient to reduce the amount of HSP-60, LPS, or other ligands that are molecularly configured to operatively interact with a TLR-4 receptor, thereby inhibiting the TLR-4 signaling pathway.

A third embodiment of the present invention is directed to a method of inhibiting TLR-4 signaling with somatic-cell gene therapy. According to this method, one administers a ribozyme-viral (adeno, adeno-associated, lentiviral or other) vector against TLR-4 mRNA in a mammal. The method utilizes a hammerhead ribozyme expression cassette in a viral backbone. Ribozymes have sequence-specific endoribonuclease activity, which makes them useful for sequence-specific cleavage of mRNAs and further inhibition of gene expression. Ribozyme therapy is widely regarded as a new and potential pharmaceutical class of reagent to treat a number of medical disorders. A desired quantity or the length of expression of the ribozyme-viral vector can be readily determined without undue experimentation, as can the most effective and convenient route of administering it. Ribozyme-viral vectors against TLR-4 mRNA permit one to uniquely assess the contribution of TLR-4 mediated cell-signaling to vascular physiology, and to therapeutically intervene in the pathology such signaling causes.

A fourth embodiment of the present invention provides a non-viral method to inhibit the expression of TLR-4. This method involves antisense therapy using oligodeoxynucleotides (“ODN”) that inhibit the expression of the TLR-4 gene product by specific base pairing of single stranded regions of the TLR-4 mRNA. The method involves synthesis of ODN complimentary to a sufficient portion of TLR-4 mRNA. The method further provides an effective amount of ODN to inhibit the TLR-4 signaling pathways in a mammal.

A fifth embodiment of the present invention provides a method to inhibit the expression of TLR-4 by RNA interference (“RNAi”). This method involves the use of double-stranded RNA (“dsRNA”) that are sufficiently homologous to a portion of the TLR-4 gene product such that the dsRNA degrades mRNA that would otherwise affect the production of TLR-4. A well-defined 21-base duplex RNA, referred to as small interfering RNA (“siRNA”), may operate in conjunction with various cellular components to silence the TLR-4 gene product with sequence homology.

A sixth embodiment of the present invention provides a method to inhibit the TLR-4 cell-signaling pathway by peptide mimetics. This method involves the introduction of small peptides (i.e., peptides of approximately 10-20 amino acids) that bind to TLR-4 ligands, thereby preventing proper TLR-4 ligands from binding to TLR-4 or associated receptors (e.g., MD2). In this manner, the TLR-4 cell-signaling pathway may be blocked from signal transduction, because the proper TLR-4 ligands are unable to bind correctly to TLR-4 or its associated receptors.

A seventh embodiment of the present invention provides a method to inhibit the expression of TLR-4 through the introduction an anti-TLR-4 antibody. Such an antibody may be delivered to a mammal through any conventional mechanism in an amount effective to inhibit the TLR-4 signaling pathways in a mammal; the mechanism of delivery and quantity of antibody necessary for inhibiting TLR-4 expression both being readily ascertainable without undue experimentation.

Other features and advantages of the invention will become apparent from the following detailed description, taken in conjunction with the accompanying drawings, which illustrate, by way of example, various features of embodiments of the invention.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 is executed in color.

FIG. 1a is a histologic depiction of TLR-4 immunoreactivity (brown) within the lipid core of an atherosclerotic plaque in the aortic sinus of an apolipoprotein E-deficient mouse.

FIGS. 1b and 1c depict the histology of macrophage (brown) and smooth muscle cell (red) immunoreactivity, respectively, in the serial section of the same aortic sinus.

FIG. 1d depicts Rabbit IgG staining for a negative control.

FIG. 1e depicts a lack of TLR-4 immunoreactivity in the non-atherosclerotic aortic mouse sinus.

FIG. 2 is executed in color, and is a series of photomicrographs indicating TLR-4 expression in human atherosclerotic lipid-rich plaques, and a lack of such expression in fibrous plaques.

FIG. 2a depicts an atherosclerotic plaque stained brown with rabbit anti-human TLR-4 antiserum.

FIG. 2b depicts a negative control where the primary antibody was replaced by rabbit IgG.

FIG. 2c depicts TLR-4 immunoreactivity (brown).

FIG. 2d depicts a double immunostain of TLR-4 (brown) and macrophages (red), demonstrating co-localization.

FIG. 2e depicts macrophage immunoreactivity (red), under a higher magnification.

FIG. 2f depicts TLR-4 immunoreactivity (brown), under a higher magnification.

FIG. 2g depicts macrophage (red) along with TLR-4 (brown) immunoreactivity, under a higher magnification.

FIG. 2h depicts a lack of immunoreactivity of TLR-4 in a fibrous plaque.

FIG. 2i depicts smooth muscle cell alpha actin immunoreactivity (red) without TLR-4 immunoreactivity (brown) upon double-staining.

FIG. 2j depicts a lack of immunoreactivity of macrophages in a fibrous plaque.

FIG. 2k depicts a negative control using pre-absorption of the antiserum with the peptide.

FIG. 2l depicts a normal mammary artery with only minimal immunoreactivity of TLR-4 along the endothelial border.

FIG. 3 is not executed in color, and depicts the relative intensity of each band, at indicated dosage levels, of TLR-4 expression when analyzed by reverse transcription polymerase chain reaction (“RT-PCR”), relative to GAPDH expression in cultured human monocyte derived macrophages that were stimulated with either native or oxidized LDL for five hours.

### DETAILED DESCRIPTION OF THE PREFERRED EMBODIMENTS

Methods of the present invention inhibit Toll-like receptor-4 (“TLR-4”) activity and expression by interfering with the production or biological activity of native TLR-4. One can use these methods to treat any disease in which inhibiting TLR-4 activity has a beneficial effect on a patient (e.g., ameliorating a disease, lessening the severity of its complications, preventing it from manifesting, preventing it from recurring, merely preventing it from worsening, or a therapeutic effort to affect any of the aforementioned, even if such therapeutic effort is ultimately unsuccessful). Diseases are known in the art in which TLR-4 activity is known or suspected to play a role in initiating, aggravating, or maintaining the pathological state that comprises the disease. Atherosclerosis, restenosis, inflammation and other vascular diseases are examples. Methods of the present invention may be used to treat any of these diseases.

In a preferred embodiment, methods of the present invention are used to inhibit atherosclerosis, transplant atherosclerosis, vein-graft atherosclerosis, stent restenosis, and angioplasty restenosis, and to thereby treat the cardiovascular diseases that atherosclerosis causes (hereinafter “vascular diseases”). These methods may be used in any patient who could benefit from reducing atherosclerosis that is already present, from inhibiting atherosclerosis that has yet to form, or from both reducing existing atherosclerosis and inhibiting new atherosclerosis. Such patients include those suffering from, for example, angina pectoris and its subtypes (e.g., unstable angina and variant angina); ischemias affecting organs such as the brain, heart, bone, and intestines, and conditions associated with the ischemias, such as stroke, transient ischemic attacks, heart attack, osteonecrosis, colitis, poor kidney function, and congestive heart failure; poor blood circulation to the extremities and the complications of poor blood circulation, such as slow wound healing, infections, and claudication; atherosclerosis itself, including restenosis following angioplasty or stenting of atherosclerotic lesions; vein-graft atherosclerosis following bypass surgery; transplant atherosclerosis; and other diseases caused by or associated with atherosclerosis.

TLR-4 may be encoded by the RNA sequence set forth herein as SEQ ID NO. 6. Inhibition of this RNA, or those substantially similar to it, may correspondingly inhibit the biological activity of TLR-4. Thus, various methods of the present invention are directed to inhibiting the expression of TLR-4 RNA.

The present invention contemplates a variety of TLR-4 inhibitors that are employed to inhibit the biological activity of TLR-4. These inhibitors may be administered to a mammal by any suitable means, such as those set forth in the various ensuing embodiments. Such inhibitors may include any compound, pharmaceutical, or other composition that affects an inhibition of the biological activity of TLR-4. Such a composition may be administered to a mammal in an effective amount and by any suitable means, including, but not limited to, orally, topically, intraveneously, intramuscularly, via a surgical device, such as a catheter, or via an implantable mechanism, such as a stent.

A first aspect of the present invention includes somatic cell gene transfer utilizing viral vectors containing TLR-4 gene sequences that express antisense RNA. Appropriate viral vectors that can express antisense TLR-4 RNA include expression vectors based on recombinant adenoviruses, adeno-associated viruses, retroviruses or lentiviruses, though non-viral vectors may be used, as well. An ideal vector for TLR-4 antisense gene transfer against atherosclerosis and angioplasty/stent-induced restenosis in mammals has the following attributes: (1) high efficacy of in vivo gene transfer; (2) recombinant gene expression in dividing as well as nondividing cells (the baseline mitotic rate in the coronary artery wall is <1% even in advanced lesions); (3) rapid and long-lived recombinant gene expression; (4) minimal vascular toxicity from inflammatory or immune responses; (5) absence of baseline immunity to the vector in the majority of the population; and (6) lack of pathogenicity of viral vectors. This is not to say that a vector must have all of these attributes; indeed, many useful vectors will not.

In a preferred embodiment of the invention, one employs adenovirus serotype 5 (“Ad5”)-based vectors (available from Quantum Biotechnology, Inc., Montreal, Quebec, Canada) to deliver and express TLR-4 gene sequences expressing antisense RNA in cultured macrophages and vascular smooth muscle cells and in atherosclerosis-prone mice and swine. The recombinant Ad5 vectors have several advantages over other vectors such as liposomes and retroviruses. Unlike retroviral vectors, proliferation of the target cell is not required for infection by adenovirus vectors and thus, Ad5 vectors can infect cells in vivo in their quiescent state. Ad5 vectors are capable of infecting a number of different tissues although the transduction efficiency can vary according to the cell type. However, Ad5 vectors as a means of in vivo gene delivery have several drawbacks: (1) gene expression from cells transduced with the Ad5 vector is often transient due to the elimination of the Ad5-transduced cells by the host immune system; (2) Ad5 vectors may generate some toxicity to human recipients as observed in human clinical trials in cystic fibrosis patients; and (3) initial administration of Ad5 vectors produces blocking antibodies to the vectors, thus repeated administrations of the adenoviral vector may not be effective. Even with these limitations, methods of the present invention utilize rAd5-mediated transfer of the TLR-4 sequence expressing antisense RNA. Using RT-PCR, a portion of TLR-4 is isolated and cloned upstream to the human cytomegalovirus (“CMV”) major immediate early promoter-enhancer in a direction to generate antisense TLR-4 RNA. The use of recombinant Ad5 vectors provides proof of the principle that adenovirus-mediated gene therapy might be particularly well suited as an adjunct to coronary angioplasty, since even temporary inhibition of smooth muscle cell proliferation might suffice to limit the formation of restenotic lesions.

A second aspect of the present invention provides a gene therapeutic method to produce high levels of soluble forms of membrane bound TLR-4 that compete for non-bound HSP-60, LPS, and other ligands that are molecularly configured to operatively interact with a TLR-4 receptor, but lack at least a substantial portion of the TLR-4 signal transduction domain.

As with other types of disease, therapeutic strategies to treat atherosclerotic disease entail treatment for an extended period of time ranging from months to years. Prolonged and efficient transgene transcription from heterologous promoters is a major consideration for gene therapies. The inclusion of a CMV promoter to drive expression of soluble TLR-4 in the present invention has been popularly used to express a variety of genes. It is, however, often subject to epigenetic silencing as are most promoters and transgenes. In an attempt to circumvent this problem, a variety of promoter expression strategies can be used to optimize the in vivo production of the soluble TLR-4 in the present invention.

Efficient gene expression in viral vectors depends on a variety of factors. These include promoter strength, message stability and translational efficiency. Each of these factors must be explored independently to achieve optimal expression of a soluble TLR-4 gene. Applications of other promoter/enhancer variants to increase and optimize the expression of soluble TLR-4 in vitro as well as in vivo are included within the scope of this invention. These include promoters or enhancers stronger than CMV that exhibit inducibilty such as tetracycline inducible promoters. Promoters/enhancers with tissue-specific functions that target, for example, vascular endothelial or smooth muscle tissue, and that produce sufficient amounts of soluble TLR-4 or its derivatives for a time and under condition sufficient to reduce the amount of TLR-4 ligand and thereby inhibit the TLR-4 function may also be included. Levels and persistence of soluble TLR-4 expression can be compared with those obtained from the CMV promoter.

A third aspect of the present invention contemplates a somatic cell gene therapeutic method by administering a ribozyme-viral (adeno, adeno-associated or lentiviral) or non-viral vector against TLR-4 mRNA in a mammal, and in particular in humans for treating the conditions referred to above. The method involves development of a hammerhead ribozyme expression cassette that targets a sequence of TLR-4 mRNA. Ribozymes are sequence-specific endoribonucleases that catalytically cleave specific RNA sequences, resulting in irreversible inactivation of the target mRNA, thereby inhibiting the gene expression. T. Cech, “Biological catalysis by RNA,” Ann Rev Biochem. 55:599-629 (1986); J. J. Rossi, “Therapeutic ribozymes: principles and applications,” Bio Drugs 9:1-10 (1998). Ribozymes offer advantages over antisense ODN. For instance, rybozymes possess higher catalytic activity than ODN; a comparatively smaller quantity of rybozyme-containing active is thus required for inhibition of gene expression. Ribozymes can be delivered exogenously or can be expressed endogenously with the use of appropriate promoters in a viral vector. Methods of the present invention utilize a hammerhead ribozyme directed to human TLR-4 mRNA. Desired quantity or the length of expression of the ribozyme-viral or non-viral vector can readily be determined by routine experimentation, as can the most effective and/or convenient route of administration.

In a fourth aspect of the present invention, there is provided a non-viral method to inhibit the expression of TLR-4. This method involves synthesis of pentadecamer (“15-mer”) ODN corresponding to the sense and antisense sequence of human TLR-4 mRNA. Pentadecamer ODN are known to bind strongly to single-stranded regions of target mRNA. D. Jaskuski et al., “Inhibition of cellular proliferation by antisense oligonucleotide to PCNA cyclin,” Science 240:1544-1548 (1988). Such strong binding may correspondingly result in strong inhibition of the translation of mRNA.

In a preferred method of the present invention, ODN are synthesized on a nucleic acid synthesizer, such as the EXPIDITE Nucleic Acid Synthesizer (available from Applied Biosystems, Inc., Rockville, Md.) and purified using standard protocols.

In a fifth aspect of the present invention, there is provided a method to inhibit the expression of TLR-4 by RNAi. This new approach to silencing a gene product by degrading a corresponding RNA sequence is reportedly more effective than alternative gene silencing methodologies, including antisense and ribozyme-based strategies. The method involves the use of dsRNA that are sufficiently homologous to a portion of the TLR-4 gene product such that the dsRNA degrades mRNA that would otherwise affect the production of TLR-4. siRNA, a well-defined 21-base duplex RNA (obtained from Dharmacon Research, Inc., Boulder, Colo.), may operate in conjunction with various cellular components to silence the TLR-4 gene product with sequence homology. RNAi is described in Hammond et al., “Post-Transcriptional Gene-Silencing by Double-Stranded RNA,” Nature 110-119 (2001); Sharp, P. A., “RNA interference—2001,” Genes Dev. 15:485-490 (2001); and Elbashir, et al., “RNA interference is mediated by 21- and 22-nucleotide RNAs,” Genes Dev. 15:188-200, each of which is incorporated by reference herein in its entirety.

Efficient gene silencing may be achieved by employing siRNA duplexes which include sense and antisense strands each including approximately 21 nucleotides, and further paired such that they possess about a 19-nucleotide duplex region and about a 2-nucleotide overhang at each 3′ terminus. Elbashir et al., “Duplexes of 21-nucleotide RNAs mediate RNA interference in cultured mammalian cells,” Nature 411:494-498 (2001). It will be appreciated by one of skill in the art of RNAi that alternately sized sense or antisense strands and/or variations on the size of the duplex and the overhang region that comprise them may be suitable for use with the methods of the present invention, and are contemplated as being within the scope thereof. Such appropriate alternate sizes may be readily ascertained without undue experimentation by one possessing such skill.

Furthermore, the inclusion of symmetric 3′-terminus overhangs may aid in the formation of specific endonuclease complexes (“siRNPs”) with roughly equivalent ratios of sense and antisense target RNA cleaving siRNPs. It is believed that the antisense siRNA strand is responsible for target RNA recognition, while the 3′-overhang in the sense strand is not involved in this function. Therefore, in a preferred embodiment, the UU or dTdT 3′-overhang of an antisense sequence is complementary to target mRNA, however the symmetrical UU or dTdT 3′-overhang of the sense siRNA oligo need not correspond to the mRNA. Deoxythymidines may be included in either or both 3′-overhangs; this may increase nuclease resistance. However, siRNA duplexes that include either UU or dTdT overhangs may be equally resistant to nuclease. The siRNA duplexes used in accordance with the present invention may be introduced to a cell via an appropriate viral or non-viral vector. Such vectors include those described above with regard to the somatic gene cell transfer embodiment of the present invention.

In a sixth aspect of the present invention, a method of inhibiting the TLR-4 cell-signaling pathway by peptide mimetics is provided. This method involves the introduction of small peptides (i.e., peptides of approximately 10-20 amino acids) that bind to TLR-4 ligands, thereby preventing these ligands from binding to a TLR-4 or associated receptor (e.g., MD2). TLR-4 is generally found on a cell surface substantially adjacent to an MD2 receptor. It is believed that, in order to initiate TLR-4 cell signal transduction, a TLR-4 ligand must bind simultaneously to the TLR-4 receptor as well as the adjacent MD2 receptor. Binding to only one of these receptors is insufficient to propagate TLR-4 cell signal transduction. Bulut et al., “Chlamydial Heat Shock Protein 60 Activates Macrophages and Endothelial Cells Through Toll-like Receptor 4 and MD2 in a MyD88-Dependent Pathway,” J. Immunol., 168:1435-1440.

The MD2 receptor is a protein constructed of approximately 133 individual amino acids. Short, overlapping segments (e.g., approximately 10-20 amino acids in length) of the MD2 receptor molecule may be separated to test which individual segments effect TLR-4 cell signal transduction by binding to a TLR-4 ligand. Segments are overlapping insofar as a portion of one end of one segment separated for testing corresponds to a portion of one end of a second segment separated for testing. Following separation, the segments are duplicated and tested to determine which comprise at least a portion of the MD2 receptor that binds to a TLR-4 ligand, such as cHSP60, LPS, or other ligands that are molecularly configured to operatively interact with a TLR-4 receptor. A segment suitable for use in accordance with the method of the present invention comprises at least a portion of the MD2 receptor that binds to a TLR-4 ligand, such that the administration of a sufficient amount of individual copies of this segment will hinder TLR-4 signal transduction. Once administered, segments preferably bind to the MD2 binding sites of the TLR-4 ligands, thereby preventing the ligands from binding to the corresponding sites on the MD2 receptor. This may significantly hinder TLR-4 cell signal transduction.

The same process may be implemented to identify a segment of the TLR-4 receptor that may similarly hinder TLR-4 signal transduction. Short, overlapping segments (e.g., approximately 10-20 amino acids in length) of the TLR-4 receptor molecule may be separated to test which individual segments effect TLR-4 cell signal transduction by binding to a TLR-4 ligand. Following separation, the segments are duplicated and tested to determine whether the segment comprises at least a portion of the TLR-4 receptor that binds to a TLR-4 ligand, such as cHSP60, LPS, or other ligands that are molecularly configured to operatively interact with a TLR-4 receptor. A segment suitable for use in accordance with the method of the present invention comprises at least a portion of the TLR-4 receptor that binds to a TLR-4 ligand, such that the administration of a sufficient amount of individual copies of this segment will hinder TLR-4 signal transduction. Once administered, segments preferably bind to the binding sites of the TLR-4 ligands, thereby preventing the ligands from binding to the corresponding sites on the TLR-4 receptor. This may significantly hinder TLR-4 cell signal transduction.

In accordance with the method of the present invention, a segment that does, in fact, include at least a portion of the MD2 or TLR-4 receptor that binds to a TLR-4 ligand may be administered to a patient. Segments that include a portion of the MD2 receptor, segments that include a portion of the TLR-4 receptor, or combinations thereof may be administered. Moreover, administration may be performed by any suitable means, including via an oral form, such as a capsule, tablet, solution, or suspension; an intravenous form; an injectable form; an implantable form, such as a stent coating, a sustained release mechanism, or a biodegradable polymer unit; or any other suitable mechanism by which an active or therapeutic agent may be delivered to a patient. The dosage may similarly be determined in accordance with the selected form of administration, the level of which may be readily ascertained without undue experimentation, as can the most suitable means of administration.

In a seventh aspect of the present invention, a method of inhibiting TLR-4 expression through the introduction an anti-TLR-4 antibody is provided. Any suitable anti-TLR-4 antibody may be used in conjunction with this aspect of the present invention, including, but in no way limited to, anti-TLR-4 antibodies, and any suitable derivatives thereof, equivalents thereof, or compounds with active sites that functions in a manner similar to anti-TLR-4 antibodies, whether those compounds are naturally occurring or synthetic (all hereinafter included within the term “anti-TLR-4 antibody”).

An appropriate quantity of an anti-TLR-4 antibody necessary to affect the method of the present invention, and the most convenient route of delivering the same to a mammal may be determined by one of ordinary skill in the art, without undue experimentation. Furthermore, it will be readily appreciated by one of such skill that an anti-TLR-4 antibody may be formulated in a variety of pharmaceutical compositions, any one of which may be suitable for use in accordance with the method of the present invention.

Such an antibody may be delivered to a mammal through any conventional mechanism in an amount effective to inhibit the TLR-4 signaling pathways in a mammal; the mechanism of delivery and quantity of antibody necessary for inhibiting TLR-4 expression both being readily ascertainable without undue experimentation.

The vascular delivery of TLR-4 inhibiting compositions composed in accordance with any of the various embodiments of the present invention can be accomplished by any of a wide range of local delivery devices and methods. K. L. March, “Methods of local gene delivery to vascular tissues,” Semin Intervent Cardiol, 1:215-223 (1996). Local delivery is preferred because, for those compositions that include a viral or non-viral vector, site-specific delivery may result in maximal therapeutic efficacy with minimal systemic side effects. These local delivery devices typically entail an endovascular or “inside-out” approach, whereby therapeutic agents are delivered to the target site via intravascular catheters or devices. Although gene transfer is demonstrated for each device, most studies of catheter-based gene transfer reveal low efficiency, rapid redistribution of the infused material, and escape of the infusate into the systemic circulation.

Recently, several devices with modified needles capable of direct injection into interstitial tissue of either myocardium or vasculature have been described. One such approach to local drug delivery is via the nipple balloon catheter, such as the INFILTRATOR® (available from InterVentional Technologies, Inc., San Diego, Calif.), although any appropriate catheter may be used. Methods of the present invention utilize the INFILTRATOR® for intramural delivery of small volumes of high-titer rAd5, where such a viral vector is appropriate. The INFILTRATOR® catheter offers improved local gene delivery by placing vector particles directly and deeply within the vascular wall. The INFILTRATOR® catheter is designed to provide direct intramural delivery of agents by mechanical access into the media and inner adventitia, which is achieved using sharp-edged injection orifices mounted on the balloon surface. P. Barath et al., “Nipple balloon catheter,” Semin Intervent Cardiol, 1:43 (1996). This catheter has been used clinically. G. S. Pavlides et al., “Intramural drug delivery by direct injection within the arterial wall: first clinical experience with a novel intracoronary delivery-infiltrator system,” Cathet Cardiovasc Diagn, 41:287-292 (1997). Further, the INFILTRATOR® has been demonstrated to yield enhanced local transduction efficiency by adenoviral vectors compared with that which may be achieved by endoluminal delivery. T. Asahara et al., “Local delivery of vascular endothelial growth factor accelerates reendothelialization and attenuates intimal hyperplasia in balloon-injured rat carotid artery,” Circulation, 91:2793-2801 (1995).

Methods of the present invention also utilize a perivascular or “outside-in” approach of drug delivery in the vessel wall by modifying the procedure applied in periadventitial carotid injury in a mouse, as described in Example 10 below. Oguchi S, et al. “Increased intimal thickening after arterial injury in hypercholesterolemic apolipoprotein E-deficient mice: finding a novel method,” Circulation (supp.) 1-548:3066 (1997); P. Dimayuga et al., “Reconstituted HDL containing human apolipoprotein A-1 reduces VCAM-1 expression and neointima formation following periadventitial cuff-induced carotid injury in apo E null mice” Biochem Biophys Res Commun. 264:465-468, (1999).

By directly targeting the genes involved via gene therapeutic approaches, methods of the present invention may be used in stent coatings that eliminate or substantially reduce restenosis following stent placement, as well as geometric miss and late in-stent thrombosis following intracoronary brachytherapy. Methods of the present invention contemplate stents coated with TLR-4 inhibiting compositions. As these gene therapeutic agents may be used as coatings on already existing stents, they may be deployed without increasing procedure time, and will not require significant additional equipment, expertise, hospitalization or expense. This strategy should prove cost-effective in the long run since, if successful, it will diminish the need for repeat hospitalizations and additional intervention procedures. Outcomes should also be favorable, to the extent that the strategy is effective in minimizing clinical events associated with restenosis following stent placement, and geometric miss and late in-stent thrombosis following intracoronary brachytherapy. Coated stents may eventually be implanted in all patients who are candidates for stents, since it is presently not possible to determine prior to the procedure which patients will suffer from restenosis or other complications associated with arterial injury following coronary intervention.

Since TLR-2 and TLR-4 play an important role in the innate immune and inflammatory response, we investigated the expression of these receptors, and found that TLR-4 exhibits preferential expression in lipid-rich and macrophage-infiltrated murine aortic and human coronary atherosclerotic plaques. The inventor's in vitro studies, described below, demonstrated basal expression of TLR-4 by macrophages, which was up-regulated by oxidized LDL (“ox-LDL”). While not wishing to be bound by any theory, these findings suggest a potential role for TLR-4 in lipid-mediated pro-inflammatory signaling in atherosclerosis. Moreover, as TLR-4 is a receptor that recognizes chlamydial antigens such as cLPS and cHSP-60, endotoxin, and other ligands that are molecularly configured to operatively interact with a TLR-4 receptor it may provide a molecular link between chronic infection, inflammation, and atherosclerosis.

The pro-inflammatory signaling receptor TLR-4 is expressed in lipid-rich, macrophage-infiltrated atherosclerotic lesions of mice and humans. Further, TLR-4 mRNA in cultured macrophages is up-regulated by ox-LDL but not native LDL (“N-LDL”). Together, these findings suggest that enhanced TLR-4 expression may play a role in inflammation in atherosclerosis.

Cells of the innate immune system, such as macrophages, have the ability to recognize common and conserved structural components of microbial origin by pattern recognition receptors. The human homologue of Drosophila Toll, TLR-4, is a pattern recognition receptor, which activates NF-κB, and up-regulates a variety of inflammatory genes in response to microbial pathogens. Toll-like receptors play a fundamental role in the activation of innate immune responses and pathogen recognition. Further, activation of NF-κB is essential for the regulation of a variety of genes involved in the inflammatory and proliferative responses of cells critical to atherogenesis. Both NF-κB and genes regulated by NF-κB are expressed in atherosclerotic lesions. Since NF-κB activation leads to transcription of a number of pro-inflammatory genes involved in athero-thrombosis, it may be that infectious agents and clamydial antigens such as LPS and/or HSP-60 contribute to enhanced and chronic inflammation by signaling through the TLR-4 receptor, which is up-regulated by ox-LDL.

The inventor's findings of increased expression of TLR-4 induced by ox-LDL suggests a potential mechanism for the synergistic effects of hypercholesterolemia and infection in acceleration of atherosclerosis observed in experimental models and human epidemiologic observations. This provides new insight into the link among lipids, infection/inflammation and atherosclerosis.

### EXAMPLE 1

### Preparation of Mouse Tissue

Five apolipoprotein E-deficient (“apoE -/-”) mice (C57BL/6J strain, aged 5 weeks, 18 to 20 grams; obtained from Jackson Laboratory, Bar Harbor, Me.) were fed a high fat, high cholesterol (i.e., atherogenic) diet containing 42% (wt/wt) fat and 0.15% cholesterol from 6 weeks of age through the duration of the experiment. After anesthesia with ETHRANE (available from Abbot Laboratories, Abbott Park, Ill.), the mice were sacrificed at 26 weeks of age, and their hearts and proximal aortas (including ascending aorta, aortic arch and a portion of descending aorta) were excised and washed in phosphate-buffered saline (“PBS”) to remove blood. The basal portion of the heart and proximal aorta were embedded in OCT compound using TISSUE-TEK VIP (available from Sakura Finetek USA, Inc., Torrance, Calif.), frozen on dry ice and then stored at −70° C. until sectioning. Serial 10 μm-thick cryosections (every fifth section from the lower portion of the ventricles to the appearance of aortic valves, every other section in the region of the aortic sinus, and every fifth section from the disappearance of the aortic valves to the aortic arch) were collected on poly-D-lysine-coated slides (available from Becton Dickinson & Co., Franklin Lakes, N.J.). Sections were stained with Oil Red O and hematoxylin, and counterstained with Fast Green (all available from Sigma Chemical Co., St. Louis, Mo., “Sigma”) for the identification of atheromatous lesions, arterial wall calcification, and cartilaginous metaplasia. The presence of calcium deposits was confirmed by the alizarin red S (available from Sigma) and von Kossa techniques using representative sections.

### EXAMPLE 2

### Preparation of Human Tissue and Human Monocyte-Derived Macrophages

Human coronary artery specimens from nine autopsy cases were collected within 24 hours of death, fixed with 10% formalin (available from Sigma) overnight and embedded in paraffin. Five of the nine coronary artery specimens included lipid-rich plaques containing a well-defined lipid-core covered by a fibrous cap, and the other four of the nine specimens included fibrous plaques, which contained mostly extracellular matrix without a lipid-core. Normal mammary artery specimens were also obtained from four additional autopsy cases. Five μm-thick sections were cut and applied to slides for both hematoxyline-eosin and immunohistochemical staining. Peripheral blood monocytes were isolated from whole blood of normal human subject by FICOLL-PAQUE density gradient centrifugation (available from Pharmacia LKB Biotechnology, Inc., Piscataway, N.J.). Monocyte-derived macrophages were cultured in RPMI 1640 (available from Sigma) containing 10% fetal calf serum (“FCS”), 100 U/ml penicillin, 100 μg/ml streptomycin and 0.25 μg/ml amphotericin B for 5 days and then starved in the culture medium without FCS but with 0.1% low endotoxin bovine serum albumin (“BSA”) (obtained from Sigma).

### EXAMPLE 3

### Immunohistochemistry

Frozen sections of the apoE -/- mouse aortic root were fixed with acetone for 5 minutes at room temperature and then immunostained with rabbit anti-hTLR-4 immune serum (1:100; obtained from Ruslan Medzhitov, Asst. Prof. of Immunobiology, Yale University, New Haven, Conn.) following the instructions on the immunostaining kit available from DAKO (Carpinteria, Calif., “DAKO”). Rat anti-mouse macros Ab (1:500; available from Serotec, U.K.) were used as macrophage marker. Colors were developed using the DAKO AES substrate system. Smooth muscle cells were stained by a mouse anti-actin Ab conjugated with alkaline phosphatase (1:50, available from Sigma). Colors were developed using VECTOR Red Alkaline Phosphatase Substrate Kit I (obtained from Vector Laboratories, Inc., Burlingame, Calif.). Rabbit IgG or rabbit serum was used as a negative control.

For human atherosclerotic plaques, following deparaffinization in graded alcohol, sections were immunostained using rabbit anti-human TLR-4 and TLR-2 antiserum (1:100) raised against extracellular peptide domains of TLR-4 and TLR-2 (available from Berkeley Antibody Company, Richmond, Calif.). Following immunoperoxidase staining, the representative fields were photographed. Cells were lysed in Laemmli buffer and separated with a 10% sodium dodecyl sulphate polyacrylamide gel electrophoresis (“SDS-PAGE”). The protein was then transferred onto a polyvinylidene difluoride membrane, and the membrane was probed with anti-TLR-2, anti-TLR-4 antibodies, and prebleeds corresponding to each antibody (1:2,000). After incubation with horseradish peroxidase-conjugated goat anti-rabbit antibody (available from Rockland Immunochemicals for Research, Gilbertsville, Pa.), the membrane was developed with an enhanced chemiluminescence ECL Western Blotting Detection Kit (available from Amersham Pharmacia Biotech UK Ltd., Buckinghamshire, England). Pre-incubating the anti-TLR-4 serum with TLR-4 peptide (SEQ ID NO. 5) was used to demonstrate specificity of the strain and rabbit IgG or rabbit serum instead of primary antibody was used as a negative control.

### EXAMPLE 4

### Double Immunohistochemistry

Double immunostaining of human atherosclerotic plaques was performed using an EnVision Doublestain System (available from DAKO). Following TLR-4 immunostaining, 3,3′-diaminobenzadine (obtained from Sigma) was used as the peroxidase chromogenic substrate. Mouse monoclonal anti-human CD68 antibody (360 μg/ml, 1:20 dilution; available from DAKO) for macrophages and mouse monoclonal anti-human α-actin antibody (100 μg/ml, 1:100 dilution; available from DAKO) for smooth muscle cells were used with Fast Red (available from Sigma) as the alkaline phosphatase chromogenic substrate.

### EXAMPLE 5

### Preparation and Modification of Lipoproteins

Human N-LDL (obtained from Sigma) was dialyzed against isotonic phosphate saline buffer (pH 7.4) to remove ethylenediamine tetraacetic acid (“EDTA”) by using a 10,000 molecular weight cut-off SLIDE-A-LYZER dialysis cassette (obtained from Pierce Chemical Co., Rockford, Ill.). Ox-LDL was prepared by incubating 0.1 mg of LDL protein/ml with 5 μM of copper sulfate (CUSO₄) for 24 hours at 37° C., and stopped by adding butylated hydroxytoluene (2,6-di-t-butyl-p-cresol) (available from Sigma) to a final concentration of 0.1 mM. Ox-LDL was separated from CuSO₄ and equilibrated into the cell culture medium over a PD-10 column (available from Pharmacia Fine Chemicals, Uppsala, Sweden). All reagents were endotoxin-free. LPS levels of LDL preparations were confirmed with a chromogenic Limulus assay and contained less than 0.3 pg of LPS/μg of LDL protein.

The extent of oxidation of the lipoprotein preparations was determined by a thiobarbituric acid reactive substance (“TBARS”) assay. Concentrated trichloroacetic acid was added to aliquots of lipoprotein samples containing 1.5 mg of protein to give a final concentration of 5%. An equal volume of 1% thiobarbituric acid was then added and the mixture was heated in a water bath at 100° C. for 20 min. After centrifugation to clarify the solution, the peak absorbance at 582 nm was read on a Beckman DB Spectrophotometer (available from Beckman Coulter, Inc., Fullerton, Calif.) against a buffer blank. The amount of thiobarbituric-reactive substance was calculated from a standard curve, with malonaldehyde bis(dimethylacetal) (available from Sigma) as the standard. The ox-LDL had 20-25 nM TBARS/mg of cholesterol.

### EXAMPLE 6

### Reverse Transcription-Polymerase Chain Reaction (RT-PCR)

Total RNA was isolated from resting N-LDL, ox-LDL stimulated human monocyte-derived macrophage cells using an RNA Stat60 isolation reagent (obtained from Tel-test ‘B’, Inc., Friendswood, Tex.) following manufacturer's instruction and treated with RNase-free DNase I. For RT reaction, the SUPERSCRIPT MMLV preamplification system (obtained from Life Technologies, Inc., Gaithersburg, Md.) was applied. PCR amplification was performed with TAQ GOLD polymerase (obtained from Perkin Elmer, Foster City, Calif.) for 32 cycles at 95° C. for 45s, 54° C. for 45s, and 72° C. for 60s (for TLR-2 and TLR-4). The oligonucleotide primers used for RT-PCR for TLR-2 were SEQ ID NO. 1 and SEQ ID NO. 2, and for TLR-4 were SEQ ID NO. 3 and SEQ ID NO. 4. Glyceraldehyde-3-phosphate dehydrogenase (“GAPDH”) primers were obtained from Clontech Laboratories, Inc. (Palo Alto, Calif.).

The TLR-2 and TLR-4 RT-PCR fragments were purified and sequenced to confirm the identity of the fragments. Real-time quantitative PCR was performed on an iCycler Thermal Cycler (obtained from Bio-Rad Laboratories, Inc., Hercules, Calif.) using an SYBR Green RT-PCR Reagents kit (obtained from Applied Biosystems, Foster City, Calif.) and the TLR primers described above. The semi-quantitative RT-PCR experiments were repeated with cells pretreated for 1 hour with 15d-PGJ₂ (20 μM), proteasome inhibitor I (100 μM) (available from Affinity Bioreagents, Inc., Golden, Colo.), or cycloheximide (10 μm/ml). Endothelial cells were pretreated with NF-κB p65 antisense and sense oligonucleotides (30 μM) for 24-48 hours, three times before LPS stimulation (50 ng/ml). For densitometry analysis, the intensity of the bands were measured by Digital Science 1D Image Analysis Software (obtained from Eastman Kodak Co., Rochester, N.Y.) and normalized with GAPDH intensity.

### EXAMPLE 7

### TLR-4 is Expressed in Atherosclerotic Lesions of the ApoE -/- Mice

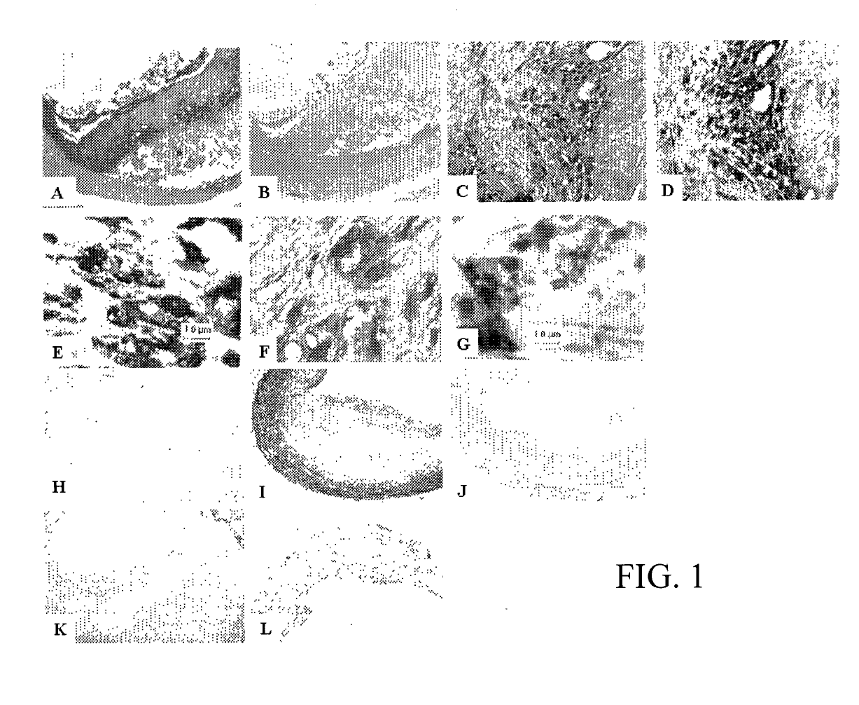

As depicted in FIG. 1, all five apoE -/- mice exhibited TLR-4 immunoreactivity in the atherosclerotic lesions of the aortic root, which co-localized with macrophage immunoreactivity. TLR-4 staining was absent in the normal vessels obtained from control C56BL/6J mice (FIG. 1e). Mouse IgG staining was negative and pre-incubation of the tissue sections with the specific peptide against which the anti-TLR-4 antiserum was generated completely blocked the TLR-4 staining in the apoE -/- vessels, indicating the specific nature of the TLR-4 immunostaining. No TLR-2 immunoreactivity was observed in normal or atherosclerotic lesions (not shown).

### EXAMPLE 8

### TLR-4 is Expressed in Human Coronary Plagues

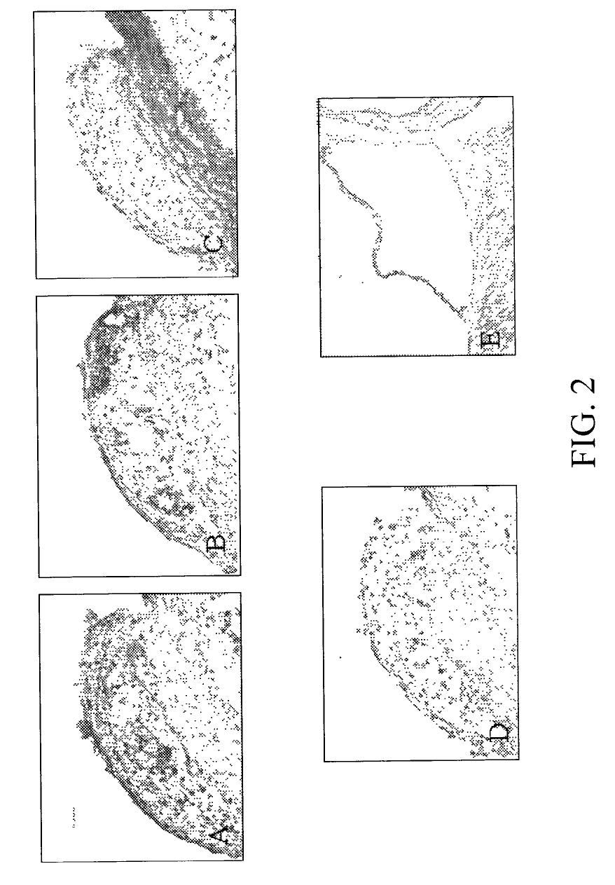

The human coronary atherosclerotic plaques were classified into lipid-rich plaques containing a well-defined lipid-core covered by a fibrous cap (n=5), and fibrous plaques which contained mostly extracellular matrix without a lipid-core (n=4). As depicted in FIG. 2, strong TLR-4 expression (brown staining) was observed around the lipid core at the shoulder of lipid-rich plaques where it co-localized with macrophage immunoreactivity. Incubation of the antiserum with the peptide used to generate the primary antibody blocked TLR-4 immunoreactivity, confirming the specificity of the anti-TLR-4 antiserum. Double staining showed close spatial co-localization of TLR-4 expression with macrophage immunoreactivity. No TLR-4 immunoreactivity or macrophage immunoreactivity was found in fibrous plaques, which demonstrated strong smooth muscle α-actin immunoreactivity. Normal mammary arteries showed only minimal or no TLR-4 expression. TLR-2 immunoreactivity was absent in all plaques while control staining was positive in THP-1 cells (not shown).

### EXAMPLE 9

### TLR-4 mRNA Regulation by Ox-LDL

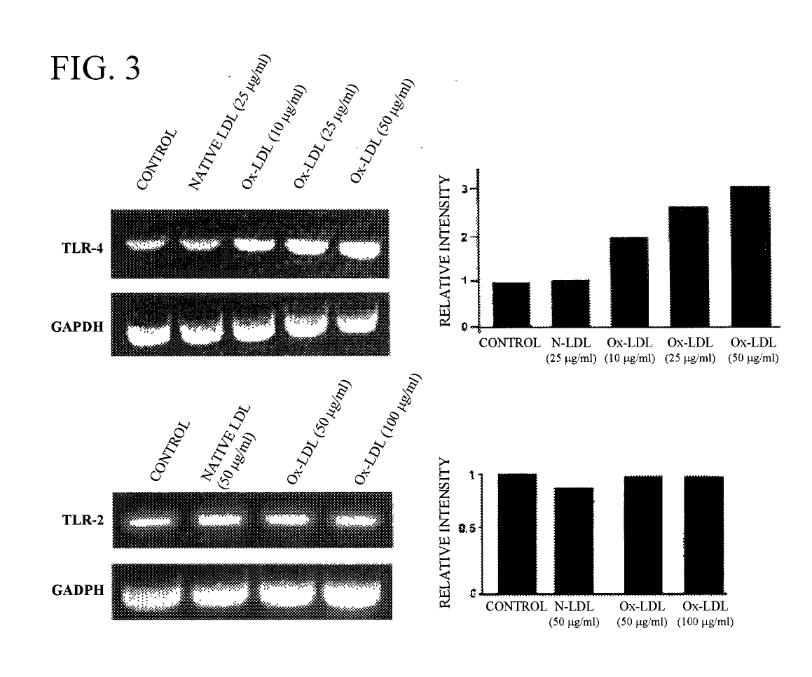

Cultured human monocyte derived macrophages were stimulated with N-LDL or ox-LDL for 5 hours. RT-PCR was performed for TLR-2 and TLR-4, and relative intensity was calculated by densitometry as described in Faure et al., at 2018-2024. As depicted in FIG. 3, RT-PCR showed basal TLR-2 and TLR-4 mRNA expression by macrophages. The TLR-4 mRNA was upregulated by ox-LDL in a dose-dependent manner and up to threefold, whereas N-LDL had no effect. TLR-2 mRNA was not upregulated by ox-LDL.

### EXAMPLE 10

### Perivascular or “Outside In” Approach to Drug Delivery

ApoE -/- mice (20 weeks of age, 6 per group) were anesthetized, and the carotid artery was exposed by making a small incision in the side of the neck. A section of artery was loosely sheathed with a cuff made of a TYGON tube (3.0 mm long, 0.5 mm inner diameter; obtained from Saint-Gobain Performance Plastics, Wayne, N.J.). A biodegradable biocompatible polymeric material, ATRIGEL (obtained from Atrix Laboratories, Ft. Collins, Colo.), a copolymer of polylactic and polyglycolic acid, was used for the local delivery of viral particles. An 18% (w/w) polymeric gel in PBS with 1×10⁸ pfu of rAd5 (right carotid) or without rAd5 (left carotid) was applied between the cuff and the vessel using a syringe and blunt cannula. The gel compound used in the study was a free-flowing liquid below body temperature. When placed in an aqueous environment at or above body temperature, the viscosity increases and the gel solidifies into a viscous mass. Once applied to the artery in vivo, the polymer turns into a gel immediately on contact and the gel is gradually resorbed in about 14 to 21 days, thereby providing potential use as a drug depot.

What is claimed is:

1. A system for inhibiting the biological activity of Toll-like receptor-4 (TLR-4) comprising:
an intravascular device; and
a therapeutic composition coated upon the intravascular device, the therapeutic composition comprising a TLR-4 inhibitor.

2. The system of claim 1, wherein the intravascular device is selected from the group consisting of a catheter and a stent.

3. The system of claim 1, wherein the TLR-4 inhibitor is selected from the group consisting of a nucleic acid expressing antisense TLR-4 RNA, a nucleic acid encoding a soluble TLR-4 protein, a nucleic acid encoding a hammerhead ribozyme that cleaves TLR-4 mRNA, an antisense TLR-4 oligodeoxinucleotide (ODN), a nucleic acid expressing a double stranded RNA (dsRNA) that is sufficiently homologous to a portion of a TLR-4 gene product such that the dsRNA is capable of inhibiting the encoding function of mRNA that would otherwise cause the production of TLR-4, a protein sequence that corresponds to at least a portion of a receptor that binds to a TLR-4 ligand during a TLR-4 signal transduction event, and an anti-TLR-4 antibody.

4. The system of claim 3, wherein the TLR-4 inhibitor is the nucleic acid expressing antisense TLR-4 RNA.

5. The system of claim 3, wherein the TLR-4 inhibitor is the nucleic acid encoding the hammerhead ribozyme that cleaves TLR-4 mRNA.

6. The system of claim 3, wherein the TLR-4 inhibitor is the antisense TLR-4 oligodeoxinucleotide (ODN).

7. The system of claim 3, wherein the TLR-4 inhibitor is the anti-TLR-4 antibody.

8. The system of claim 1, wherein the TLR-4 inhibitor is included within a vector.

9. The system of claim 8, wherein the vector is selected from the group consisting of adenoviruses, adeno-associated viruses, retroviruses, lentiviruses, viral vectors, and non-viral vectors.

10. The system of claim 8, wherein the vector is an adenovirus serotype 5-based vector.

11. The system of claim 8, wherein the TLR-4 inhibitor is selected from the group consisting of a nucleic acid expressing antisense TLR-4 RNA, a nucleic acid encoding soluble TLR-4 protein, a nucleic acid encoding a hammerhead ribozyme that cleaves TLR-4 mRNA, and a nucleic acid expressing a double stranded RNA (dsRNA) that is sufficiently homologous to a portion of a TLR-4 gene product such that the dsRNA is capable of inhibiting the encoding function of mRNA that would otherwise cause the production of TLR-4.

12. The system of claim 1, further comprising an amount of the therapeutic composition sufficient to inhibit a vascular disease.

13. The system of claim 12, wherein the vascular disease is selected from the group consisting of atherosclerosis, transplant atherosclerosis, vein-graft atherosclerosis, thrombosis, restenosis, stent restenosis, and angioplasty restenosis.

14. The system of claim 3, wherein the TLR-4 inhibitor is the nucleic acid encoding the soluble TLR-4 protein.

15. The system of claim 14, wherein the soluble TLR-4 protein is unable to participate in normal TLR-4 signal transduction.

16. The system of claim 14, wherein the soluble TLR-4 protein lacks a substantial portion of the normal TLR-4 signal transduction domain.

17. The system of claim 14, wherein the soluble TLR-4 protein competes for a non-bound TLR-4 ligand.

18. The system of claim 17, wherein the non-bound TLR-4 ligand is a chlamydial heat shock protein-60 (cHSP60) or a lipopolysaccharide (LPS).

19. The system of claim 3, wherein the TLR-4 inhibitor is the nucleic acid expressing the dsRNA, and the dsRNA further includes:
a sense strand further including approximately 21 nucleotides; and
an antisense strand further including approximately 21 nucleotides.

20. The system of claim 19, wherein the sense strand and the antisense strand are paired such that they possess a duplex region of approximately 19 nucleotides.

21. The system of claim 19, wherein the sense strand and the antisense strand each further include an overhang at a 3′-terminus of approximately 2 nucleotides.

22. The system of claim 21, wherein the sense overhang and the antisense overhang are symmetrical.

23. The system of claim 21, wherein the antisense overhang comprises a UU 3′-overhang or a dTdT 3′-overhang.

24. The system of claim 23, wherein the UU 3′-overhang or the dTdT 3′-overhang is complementary to the mRNA.

25. The system of claim 21, wherein at least one of the sense overhang and the antisense overhang further includes a deoxythymidine.

26. The system of claim 3, wherein the TLR-4 inhibitor is the protein sequence that corresponds to at least the portion of the receptor that binds to the TLR-4 ligand during the TLR-4 signal transduction event.

27. The system of claim 26, wherein the receptor is a TLR-4 receptor or an MD2 receptor.

28. The system of claim 26, wherein the protein sequence comprises from about 10 to about 20 amino acids.

29. A method of treating a vascular disease, the method comprising the steps of:
providing a TLR-4 inhibitor; and
administering the TLR-4 inhibitor to a mammal in an amount effective to at least partially inhibit the biological activity of TLR-4.

30. The method of claim 29, wherein the vascular disease is selected from the group consisting of atherosclerosis, transplant atherosclerosis, vein-graft atherosclerosis, thrombosis, restenosis, stent restenosis, and angioplasty restenosis.

31. The method of claim 29, wherein the step of administering the TLR-4 inhibitor further comprises administering the TLR-4 inhibitor in an amount effective to inhibit the vascular disease.

32. The method of claim 29, wherein the step of administering the TLR-4 inhibitor further comprises administering the TLR-4 inhibitor intraveneously.

33. The method of claim 29, wherein the step of administering the TLR-4 inhibitor further comprises administering the TLR-4 inhibitor intramuscularly.

34. The method of claim 29, wherein the step of administering the TLR-4 inhibitor further comprises delivering the TLR-4 inhibitor with an intravascular device.

35. The method of claim 34, wherein the intravascular device is a catheter or a stent.

36. The method of claim 34, wherein the intravascular device is coated with the TLR-4 inhibitor.

37. The method of claim 29, wherein the TLR-4 inhibitor is selected from the group consisting of a nucleic acid expressing antisense TLR-4 RNA, a nucleic acid encoding a soluble TLR-4 protein, a nucleic acid encoding a hammerhead ribozyme that cleaves TLR-4 mRNA, an antisense TLR-4 oligodeoxinucleotide (ODN), a nucleic acid expressing a double stranded RNA (dsRNA) that is sufficiently homologous to a portion of a TLR-4 gene product such that the dsRNA is capable of inhibiting the encoding function of mRNA that would otherwise cause the production of TLR-4, a protein sequence that corresponds to at least a portion of a receptor that binds to a TLR-4 ligand during a TLR-4 signal transduction event, and an anti-TLR-4 antibody.

38. The method of claim 37, wherein the TLR-4 inhibitor is the nucleic acid expressing antisense TLR-4 RNA.

39. The method of claim 37, wherein the TLR-4 inhibitor is the nucleic acid encoding the hammerhead ribozyme that cleaves TLR-4 mRNA.

40. The method of claim 37, wherein the TLR-4 inhibitor is the antisense TLR-4 oligodeoxinucleotide (ODN).

41. The method of claim 37, wherein the TLR-4 inhibitor is the anti-TLR-4 antibody.

42. The method of claim 37, wherein the TLR-4 inhibitor is included within a vector.

43. The method of claim 42, wherein the vector is selected from the group consisting of adenoviruses, adeno-associated viruses, retroviruses, lentiviruses, viral vectors, and non-viral vectors.

44. The method of claim 42, wherein the vector is an adenovirus serotype 5-based vector.

45. The method of claim 42, wherein the TLR-4 inhibitor is selected from the group consisting of a nucleic acid expressing antisense TLR-4 RNA, a nucleic acid encoding soluble TLR-4 protein, a nucleic acid encoding a hammerhead ribozyme that cleaves TLR-4 mRNA, and a nucleic acid expressing a double stranded RNA (dsRNA) that is sufficiently homologous to a portion of a TLR-4 gene product such that the dsRNA is capable of inhibiting the encoding function of mRNA that would otherwise cause the production of TLR-4.

46. The method of claim 37, wherein the TLR-4 inhibitor is the nucleic acid encoding the soluble TLR-4 protein.

47. The method of claim 46, wherein the soluble TLR-4 protein is unable to participate in normal TLR-4 signal transduction.

48. The method of claim 46, wherein the soluble TLR-4 protein lacks a substantial portion of the normal TLR-4 signal transduction domain.

49. The method of claim 46, wherein the soluble TLR-4 protein competes for a non-bound TLR-4 ligand.

50. The method of claim 49, wherein the non-bound TLR-4 ligand is a chlamydial heat shock protein-60 (cHSP60) or a lipopolysaccharide (LPS).

51. The method of claim 37, wherein the TLR-4 inhibitor is the nucleic acid expressing the dsRNA, and the dsRNA further includes:
a sense strand further including approximately 21 nucleotides; and
an antisense strand further including approximately 21 nucleotides.

52. The method of claim 51, wherein the sense strand and the antisense strand are paired such that they possess a duplex region of approximately 19 nucleotides.

53. The method of claim 52, wherein the sense strand and the antisense strand each further include an overhang at a 3′-terminus of approximately 2 nucleotides.

54. The method of claim 53, wherein the sense overhang and the antisense overhang are symmetrical.

55. The method of claim 53, wherein the antisense overhang comprises a UU 3′-overhang or a dTdT 3′-overhang.

56. The method of claim 55, wherein the UU 3′-overhang or the dTdT 3′-overhang is complementary to the mRNA.

57. The method of claim 53, wherein at least one of the sense overhang and the antisense overhang further includes a deoxythymidine.

58. The method of claim 37, wherein the TLR-4 inhibitor is the protein sequence that corresponds to at least the portion of the receptor that binds to the TLR-4 ligand during the TLR-4 signal transduction event.

59. The method of claim 58, wherein the receptor is a TLR-4 receptor or an MD2 receptor.

60. The method of claim 58, wherein the protein sequence comprises from about 10 to about 20 amino acids.
